(19) United States
(12) Patent Application Publication
(10) Pub. No.: US 2025/0099724 A1
(43) Pub. Date: Mar. 27, 2025

(54) SYSTEMS, METHODS AND DEVICES FOR ADAPTIVE ANGIOPLASTY BALLOON INFLATION AND DEFLATION

(71) Applicant: CARDIOVASCULAR SYSTEMS, INC., St. Paul, MN (US)

(72) Inventor: Victor L. Schoenle, Greenfield, MN (US)

(21) Appl. No.: 18/690,570

(22) PCT Filed: Jun. 30, 2022
(86) PCT No.: PCT/US2022/073283

§ 371 Date: Mar. 8, 2024

Related U.S. Application Data
(60) Provisional application No. 63/242,532, filed on Sep. 10, 2021.

(51) Int. Cl. A61M 25/10; G16H 40/63

(52) U.S. Cl. CPC A61M 25/104; A61M 25/10184; G16H 40/63; A61M 2205/3327; A61M 2205/3344; A61M 2205/3379; A61M 2205/50

(57) ABSTRACT

Various embodiments of the systems, methods and devices are provided comprising angioplasty to break up calcification or other tissue in occlusive areas within a blood vessel with automated adaptation to volume and/or pressure data that is outside of reaction windows having predetermined boundaries. The various embodiments disclosed comprise pressure pulse periods designed to break up calcified occlusive material through a cyclically stretching of the vessel walls without damaging the vessel wall tissue.

### CROSS-REFERENCE TO RELATED APPLICATIONS

This application claims the benefit of U.S. Provisional Patent Application No. 63/242,532, filed Sep. 10, 2021 and titled SYSTEMS, METHODS AND DEVICES FOR ADAPTIVE ANGIOPLASTY BALLOON INFLATION AND DEFLATION, the entire content of which is incorporated herein by reference.

### STATEMENT REGARDING FEDERALLY SPONSORED RESEARCH OR DEVELOPMENT

Not Applicable

### BACKGROUND OF THE INVENTION

### Field of the Invention

The invention relates to systems, devices and methods for breaking up calcified lesions in an anatomical conduit. More specifically, specific incremental pressure increases are provided to a balloon within a calcified conduit, e.g., a blood vessel, to break the calcified material while not damaging the tissue of the vessel wall.

### Description of the Related Art

A variety of techniques and instruments have been developed for use in the removal or repair of tissue in arteries and similar body passageways. A frequent objective of such techniques and instruments is the removal of atherosclerotic plaque in a patient's arteries. Atherosclerosis is characterized by the buildup of fatty deposits (atheromas) in the intimal layer (i.e., under the endothelium) of a patient's blood vessels. Very often over time what initially is deposited as relatively soft, cholesterol-rich atheromatous material hardens into a calcified atherosclerotic plaque. Such atheromas restrict the flow of blood, and therefore often are referred to as stenotic lesions or stenoses, the blocking material being referred to as stenotic material. If left untreated, such stenoses can cause angina, hypertension, myocardial infarction, strokes and the like.

Angioplasty, or balloon angioplasty, is an endovascular procedure to treat by widening narrowed or obstructed arteries or veins, typically to treat arterial atherosclerosis. A collapsed balloon is typically passed through a pre-positioned catheter and over a guide wire into the narrowed occlusion and then inflated to a fixed size. The balloon forces expansion of the occlusion within the vessel and the surrounding muscular wall until the occlusion yields from the radial force applied by the expanding balloon, opening up the blood vessel with a lumen inner diameter that is similar to the native vessel in the occlusion area and, thereby, improving blood flow.

The angioplasty procedure does present some risks and complications, including but not limited to: arterial rupture or other damage to the vessel wall tissue from over-inflation of the balloon catheter, the use of an inappropriately large or stiff balloon, or the presence of a calcified target vessel; and/or hematoma or pseudoaneurysm formation at the access site. As described above, the primary problem with known angioplasty systems and methods is that the occlusion yields over a relatively short time period at high stress and strain rate, often resulting in damage or dissection of the conduit, e.g., blood vessel, wall tissue.

Currently, the best way to deal with the high stress strain of blood vessel, e.g., artery, wall tissue adjacent to calcified occlusions is to use an atherectomy system marketed by Cardiovascular Systems, Inc., (“CSI”) assignee of the instant application. This system comprises an abrasive crown mounted on the drive shaft, wherein the abrasive crown is “eccentric.” i.e., with a center of mass located radially away from the drive shaft's axis of rotation. This eccentric (or non-concentric) crown sands and removes calcium internal to the intimal layer of the subject vessel wall in combination with impact energy from the orbiting rotational eccentric crown which works to break and/or soften the embedded calcified plaque.

The CSI atherectomy system and method typically increases the compliance of the calcified occlusion. This is confirmed by balloon inflations requiring lower inflation pressures post atherectomy procedure than non-atherectomy cases. However, the CSI atherectomy system and method may still the use of an adjunctive dilatation balloon to improve lumen diameter gain at the occlusion when there is calcium present within the intimal wall, i.e., not located within the vessel lumen.

Certain angioplasty balloon devices may be operated manually, wherein the inflation and deflation operations are executed by a medical professional. In other cases, at least some of the inflation and/or deflation operations may be executed according to programmed instructions that are stored within a memory and read and executed by a processor that drives inflation and/or deflation according to the programmed instructions.

It would be highly desirable to provide an inflation device which may be used to adaptively inflate and/or deflate angioplasty balloons. Various embodiments of the present invention described herein address sensing of certain parameters and the adaptation of inflation and/or deflation operations based at least in part on the sensed parameters.

Moreover, we provide disclosure of the following patents and applications, each of which are assigned to Cardiovascular Systems, Inc., and incorporated herein in their entirety, each of which may comprise systems, methods and/or devices that may be used with various embodiments of the presently disclosed subject matter:

U.S. patent application Ser. No. 13/624,313, “ROTATIONAL ATHERECTOMY DEVICE WITH ELECTRIC MOTOR”;

U.S. patent application Ser. No. 14/315,774, “DEVICES, SYSTEMS AND METHODS FOR LOCALLY MEASURING BIOLOGICAL CONDUIT AND/OR LESION COMPLIANCE, OPPOSITION FORCE AND INNER DIAMETER OF A BIOLOGICAL CONDUIT”;

U.S. patent application Ser. No. 14/801,269, “METHODS, DEVICES AND SYSTEMS FOR SENSING, MEASURING AND/OR CHARACTERIZING VESSEL AND/OR LESION COMPLIANCE AND/OR ELASTANCE CHANGES DURING VASCULAR PROCEDURES”; AND

U.S. Pat. No. 10,898,214, “SYSTEMS, METHODS AND DEVICES FOR PROGRESSIVELY SOFTENING MULTI-COMPOSITIONAL INTRAVASCULAR TISSUE”.

Various embodiments of the present invention address the issues, among others, discussed above.

### BRIEF DESCRIPTION OF THE SEVERAL VIEWS OF THE DRAWINGS

FIG. 1 is a graphic illustration of a typical stress strain curve of a single balloon inflation to the point where the artery wall tissue is damaged.

FIG. 2 is a graphic indicating that arteries with higher collagen content will be softened to a greater degree than arteries with lower collagen content.

FIG. 3 is a graphic illustrating that different arteries have different collagen to elastin ratios.

FIG. 4 is a pressure plot obtained using one embodiment of the present invention.

FIG. 5 is a graphic illustration of balloon diameter change in conjunction with the pressures employed in the embodiment of the present invention giving rise to the pressure plot of FIG. 4.

FIG. 6 illustrates a schematic view of one embodiment of the present invention.

FIG. 7 illustrates a schematic view of one embodiment of the present invention.

FIG. 8A illustrates a pressure plot for an embodiment of the present invention.

FIG. 8B illustrates a diameter plot for the embodiment of FIG. 8A.

FIG. 8C illustrates a pressure plot for another embodiment of the present invention.

FIG. 8D illustrates a diameter plot for the embodiment of FIG. 8C.

FIG. 9 is a plot of pressure, volume and balloon diameter for an inflating balloon within three exemplary arteries.

FIG. 10 is a plot of pressure, diameter and volume for an exemplary balloon.

FIG. 11 is a plot of pressure, diameter and volume for an inflating balloon within a healthy artery.

FIG. 12 is a plot of pressure, diameter and volume for an inflating balloon within an exemplary lesion.

FIG. 13 is a cutaway view of an exemplary deflated balloon within a lesion disposed along a blood vessel.

FIG. 14 is a cutaway view of an exemplary deflated balloon within a lesion disposed along a blood vessel.

FIG. 15 is a plot of exemplary monitoring of balloon pressure and volume data, together with related reaction windows.

### DETAILED DESCRIPTION OF THE INVENTION

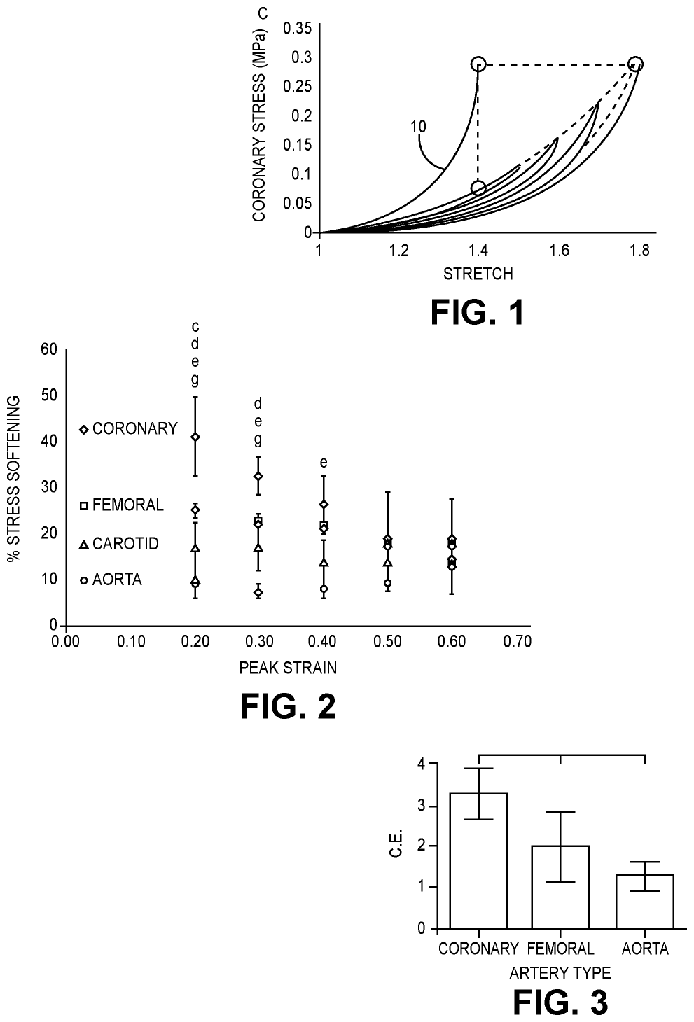

Various embodiments of balloon angioplasty devices, methods and results are illustrated in the Figures. Thus, FIG. 1 is a graphic illustration comprising a reference line 10 illustrating the typical stress strain curve of a single balloon inflation procedure to the point where the artery wall is damaged. The remaining lines, and dots, illustrate how a pulsatile inflation/cyclically stretched pressure pulse period serially applied as described herein lowers the applied stress for a given strain on the artery wall and/or may be strained further at similar safe stress levels.

FIG. 2 is a graphic indicating that arteries with higher collagen content will be softened to a greater degree than arteries with lower collagen content. FIG. 3 is a graphic illustrating that different arteries have different collagen to elastin ratios.

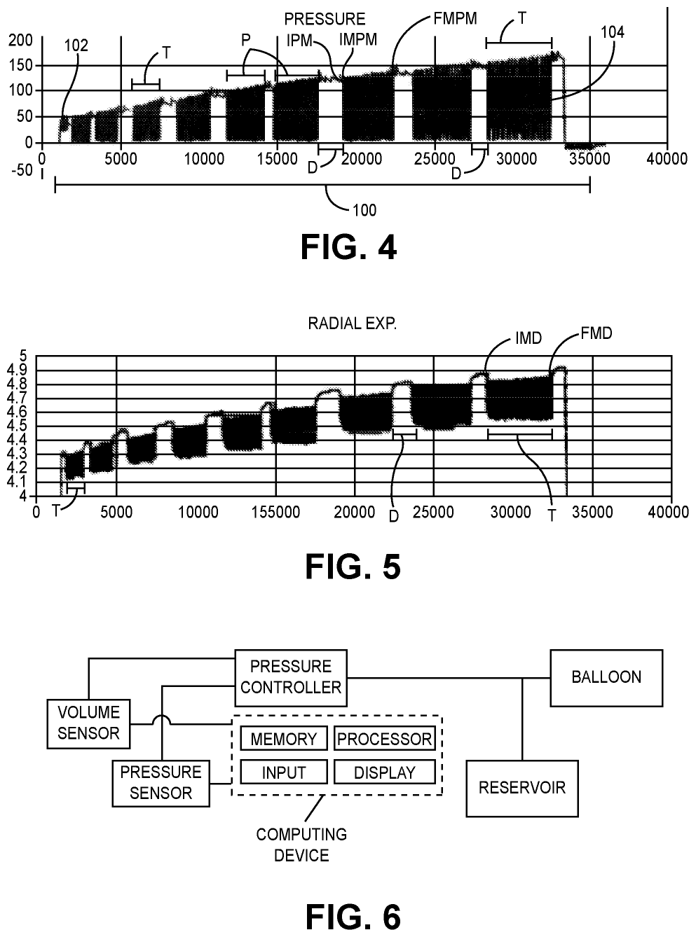

FIG. 4 is a pressure plot obtained using one embodiment of the present invention in a cadaver study. The method creates a successive series of pressure pulse periods with 40 steps per atmosphere wherein the velocity (strain rate) was set to a unit less number of 15. The steps may be modified to any number, e.g., 1 to 99 steps and the velocity may also be modified to any number, e.g., from 1 to 99.

FIG. 5 is a graphic illustration of balloon diameter change in conjunction with the pressures employed in the embodiment of the present invention giving rise to the pressure plot of FIG. 4. The balloon diameter changes are driven by the material properties and will vary between manufacturers and models of the various known balloons.

Thus, certain embodiments of the present invention comprise a plurality of pressure pulse periods, with relaxation periods therebetween, delivered via a balloon placed within an occlusion within a biological conduit, e.g., a blood vessel such as an artery. Each pressure pulse period comprises a beginning timepoint with an initial minimum pressure magnitude (IMPM) and an ending timepoint with a final maximum pressure magnitude (FMPM). The pressure pulse periods may increase, or vary, pressure magnitude within each pressure pulse period and/or may comprise a single magnitude pressure magnitude within each pressure pulse period. In addition, the time interval for each pressure pulse period may successively increase from an initial pressure pulse period time interval to a final pressure pulse period time interval, as shown in FIG. 5. Alternatively, the time intervals T for the pressure pulse period applications may be substantially equivalent in certain embodiments. Further, the pressure pulse periods may increase in magnitude from an initial pressure pulse period 102 to a final pressure pulse period 104 as is best illustrated in FIG. 4. In addition, the pressure magnitude within an individual pressure pulse period may be constant or may increase, or otherwise be variable. For example, each pressure pulse period may comprise a successively increasing plurality of pressure magnitudes between the initial minimum pressure magnitude (IMPM) and the final maximum pressure magnitude (FMPM). An example of increasing pressure magnitude within individual pressure pulse periods is shown in FIGS. 4 and 5, with 5 illustrating the related radial expansion of the balloon as referenced by the y-axis. As shown in FIG. 4, each pressure pulse period may further comprise an initiation pressure magnitude (IPM) adapted to initiate a successive pressure pulse period, with the initiation pressure magnitude (IPM) being greater than zero and less than the final maximum pressure magnitude of the immediately preceding pressure pulse period in the series of pressure pulse periods. In such examples, the final maximum pressure magnitude increases across the series of pressure pulse periods (e.g., with the final maximum pressure magnitude of at least one successive pulse period being greater than the final maximum pressure magnitude (FMPM) of each preceding pressure pulse period). In addition, the initial minimum pressure magnitude (IMPM) of at least one successive pulse period may be greater than the initial minimum pressure magnitude of each preceding pressure pulse period, as shown in FIG. 4.

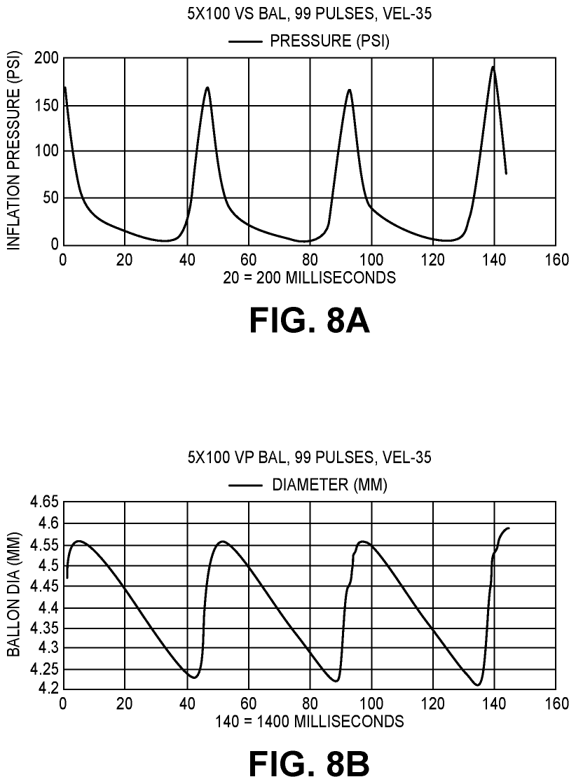

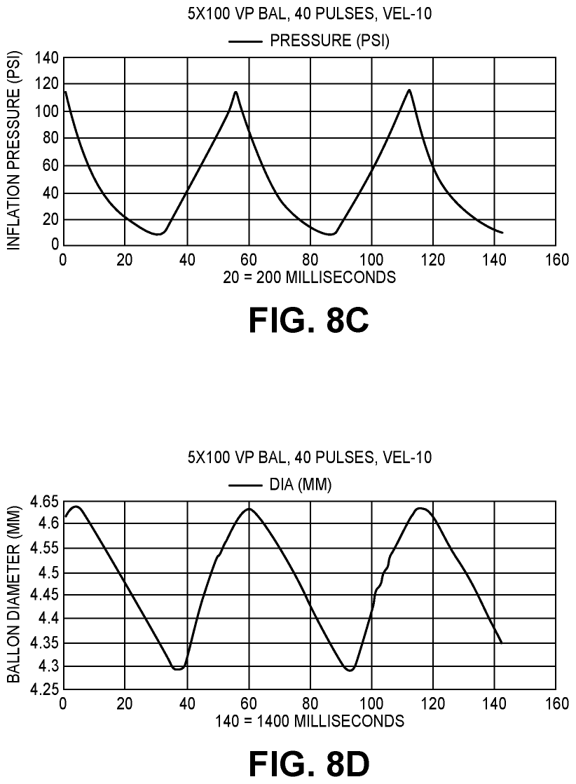

Accordingly, and with reference to FIGS. 4, 5 and 8A-8D, a method according to certain embodiments of the present invention comprise a series 100 of pressure pulse periods P applied to the internal walls of a blood vessel over a period of time, each pressure pulse period P comprising a time T that may be constant or may vary, e.g., increase with each successive pressure pulse period P within the series of pressure pulse periods 100. Each pressure pulse period P may comprise balloon inflation(s) comprising at least one pressure wave form, a pressure magnitude or magnitudes within each individual pressure wave form and/or across the pressure pulse period comprising one or more pressure wave forms. The pressure magnitude is represented in FIG. 4 by the y-axes, with time on the x-axis. The pressure magnitude for each pressure wave form may be constant within the wave form or may vary, e.g., may increase with time. Alternatively, or in combination with the pressure magnitude, the balloon's radial expansion may be a further element of the pressure pulse period(s) as illustrated by the y-axis in FIG. 5, as defined by an initial minimum diameter (IMD) and a final maximum diameter (FMD) for the balloon during each pressure pulse period. Further, each pressure wave form may comprise a time of pressuring 102 that may be constant or that may vary across the pressure wave forms of the series of pressure pulse periods. Moreover, a decompression, or relaxation, period between each successive or adjacent pressure wave forms D is provided to allow the vessel material time to relax and realign. As illustrated in FIG. 4, a decompression, or relaxation, period between successive pressure pulse periods may comprise at least one pressure magnitude within the balloon that is greater than zero. The length in time of the decompression/relaxation periods may be equal through the series of pressure pulse periods or may be variable. Finally, with particular reference to FIGS. 8A-8D, the velocity of the pressure increase, i.e., balloon inflation, at the beginning of an individual pressure pulse period, and the velocity of the pressure decrease, i.e., balloon deflation, at the end of an individual pressure pulse period are significant elements of the series of pressure pulse periods.

It will be understood that the series of pressure pulse periods 100, and all elements and variables comprising the series of pressure pulse periods 100 may be predetermined and executed using a controller comprising a processor capable of executing programmed instructions that, when executed, result in a balloon expansion regimen that follows the series of pressure pulse periods 100.

Examples of pressure pulse period series 100 are provided in FIGS. 4, 5 and 8A-8D. FIGS. 8A-8D illustrate some exemplary wave forms that may be used to achieve the intended results of the present invention. However, pulses, velocities and waveforms used in various embodiments of the present invention may vary, as shown in FIGS. 8A-8D. For example, wave forms may be non-variable in shape, for example a repeating constant pressure such as a sine wave of constant peak magnitude and period (time), or may be variable, i.e., with varying pressure and/or period. In addition, the pressure waveform types may be the same, e.g., all sine waves, within a particular pressure pulse period P, or the waveforms may vary within a pressure pulse period P. e.g., sine waves alternating with square waves and/or triangle waves or saw tooth waves as the skilled artisan will readily recognize. Similarly, the waveform types may be constant, or may vary across the series of pressure pulse periods 100 so that one pressure pulse period P in the series of pressure pulse periods 100 employs square waves and a second pressure pulse period P in the series of pressure pulse periods 100 employs saw tooth waves. The skilled artisan will recognize equivalents of these parameters, all of which are within the scope of the present invention.

Thus, the balloon outer diameter is systematically increased and decreased, at specified velocities, by predetermined specific pressure increments over predetermined time intervals. The exemplary vessel, e.g., arterial, wall is given time to relax between each pressure pulse period application. The cyclic nature of longer and longer strains through each successive pressure pulse period as shown in FIGS. 4 and 5 causes weaker short chains of vessel wall material to disengage giving the longer and more entangled chains of vessel wall material time to align and conform to the strain being applied in a way that causes less overall vessel wall material chain breakage and resulting tissue damage. Stated differently, the pressure magnitude for each pressure pulse period is selected so as to not deform the subject vessel wall non-elastically. Because a preferred embodiment of the present invention comprises an incremental increase in at least one of the variable elements, e.g., pressure magnitude, time of pressure application, velocity of pressure, etc., the vessel wall is allowed to adapt to the increasing load without deformation while the balloon breaks up calcified material.

Because the longer and more entangled vessel wall material chains are not broken or damaged, the exemplary artery may be strained further at safe stress levels, or the artery may be strained to similar pressure levels as known angioplasty methods, but with lower stress levels placed on the vessel wall over the length of the inventive procedure, resulting in lower overall vessel wall material chain/tissue damage.

In addition to the stress softening advantages with reduction of tissue damage, including reduction in cell injury responses, there is another benefit. That is, the expanded section of conduit, e.g., a blood vessel such as an artery, that has been stress softened will have increased compliance. This, in turn, results in healthy normal conduit, e.g., artery, compliance with normal blood pressure returning to the previously compromised artery.

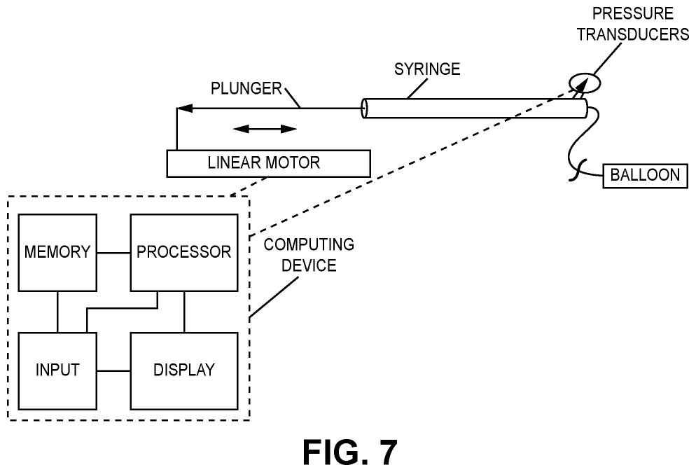

The angioplasty methods and results illustrated in FIGS. 1-5 may all be achieved with a pre-programmed system comprising a memory and processor. Sec, e.g., FIGS. 6 and 7.

FIG. 6 illustrates an exemplary system for implementing the pressure pulse periods of the various embodiments of the present invention. Thus, a pressure controller having programmed instructions therein and/or otherwise adapted to provide the pressure pulse periods in a predetermined sequence as described above is provided. The pressure controller is operatively connected, either wired or wirelessly, to a fluid reservoir and to a known balloon capable of fluid inflation from the reservoir according to the instructions provided by the pressure controller.

The system of FIG. 6 is further shown in operative communication with an external computing device comprising a memory in communication with a processor and an input, e.g., keyboard that is also in operative communication with the processor and a display which is, in turn, in operative communication with the processor. As the skilled artisan will recognize, the memory may store programmed instructions for the series of pressure pulse periods 100 and the processor may be adapted to execute the stored programmed instructions. A volume sensor and pressure sensor are shown in operative communication with the pressure controller and with the computing device for receiving and transmitting volume and pressure data to the system.

Still more alternatively, a pressure controller that functions in a manner similar to a speaker coil in order to change the pressure wave form at a wider/higher range of frequencies with a wide amplitude range and with more precision may be employed to generate the desired pressure pulse periods of the present invention.

The functionality of the above method may be achieved using a variety of devices including as shown in FIG. 6. Alternatively, as in FIG. 7, the system may comprise a balloon of known elasticity, or compliance, a device, e.g., a syringe, that is capable of injecting a known and fixed volume of fluid to inflate the balloon to the required pressure pulse period requirements, an optional pressure transducer in operative communication and connection with the inflating balloon to measure the pressure experienced by the balloon as it inflates. There is illustrated an exemplary linear motor that is capable of translating the plunger of syringe to meet the pressure pulse period requirements. A pressure transducer, when present, is in operative communication and connection with the balloon to measure and display and/or record the pressure data as well as the corresponding volume data.

The system of FIG. 7 is shown in operative communication with an external computing device comprising a memory in communication with a processor and an input, e.g., keyboard that is also in operative communication with the processor and a display which is, in turn, in operative communication with the processor. As the skilled artisan will recognize, the memory may store programmed instructions for the series of pressure pulse periods 100 and the processor may be adapted to execute the stored programmed instructions.

Still more alternatively, a pressure controller that functions in a manner similar to a speaker coil in order to change the pressure wave form at a wider/higher range of frequencies with a wide amplitude range and with more precision may be employed to generate the desired pressure pulse periods of the present invention.

Beyond the pre-programmed inflation and deflation methods and devices described above, the inventor has discovered mechanisms and methods to further adapt the inflation/deflation method and/or algorithms based on sensed or measured data or parameters collected during inflation of a typical balloon catheter. The adaptation may be achieved manually in some embodiments, but in a preferred embodiment, the adaptation is executed automatically.

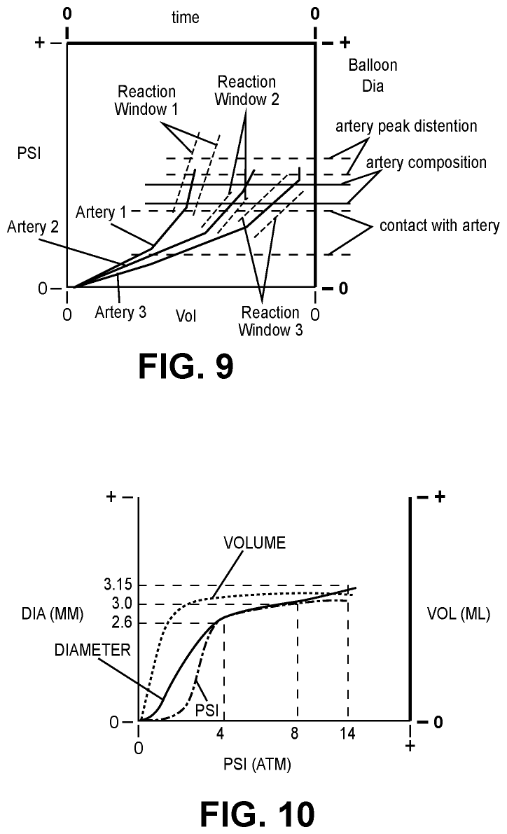

Generally then, with reference to FIG. 9, there are four main stages that a typical balloon in known angioplasty procedures may encounter.

### First Stage

The first stage is the inflation line having a slope originating from the origin where Pressure per Square Inch (PSI) and Volume (Vol) are both at zero (0), then moving upward as both PSI and Vol. increase. This initial stage represents the unbounded inflation of a balloon of a specific type and size in 37 degree C. water and generally provides a linear relationship between PSI and Volume and is referred to herein as an inflation line. The first stage ends at the lower horizontal dashed line which marks the lower boundary of the “contact with artery” region. Relative volume and/or displacement may be derived from the motion and/or position of the linear actuator. In some embodiments there is an intermediate pressure plateau prior to stage 2 while the balloon fills at substantially constant pressure.

### Second Stage

The second stage is shown within the two lower-most horizontal dashed lines covering the region labeled “contact with artery”. The artery's natural distension curve creates more resistance to expansion of the balloon (measured in diameter). This added resistance to expansion of the balloon creates, in turn, an increase in the slope of the inflation line, as compared with the slope of the first stage “balloon only” inflation line. There is variability in the resistance to expansion in various arteries.

This is illustrated in the differences in which the slopes change during the second stage for 3 types of arteries, arteries 1, 2 and 3. The left-most inflation line corresponds with artery 1 in the “contact with artery” region and has a slope that rises immediately after the inflation line enters the region, while the middle inflation line corresponding with artery 2 and the right-most inflation line corresponding with artery 3 continue along the same general slope for a period of time as was experienced in the first stage. However, both the middle inflation line for artery 2 and the right-most inflation line for artery 3 experience an increase in slope as the artery resistance exerts its effect on the balloon's expansion. The middle inflation line slope for artery 2 turns upward first in time compared with that of the right-most inflation line for artery 3. This indicates that artery 2 of the middle inflation line begins exerting meaningful resistance against balloon expansion sooner in time than that of artery 2 associated with the right-most inflation line. And, the inflation line of artery 1 begins to exert meaningful resistance against balloon expansion immediately, or nearly immediately, upon contacting artery 1 with the expanding balloon. It has been observed that different regions of a stenosis present different elasticity and diameter resulting in different patterns of pressure and volume change as the balloon contacts and begins to deform the artery.

### Third Stage

The third stage is the next set of dashed lines covering a region labeled “artery composition”. In this stage, the inflation slope may change further (steepen), as is seen in both the left-most inflation line (artery 1) and the middle inflation line (artery 2). However, the right-most inflation line (artery 3) maintains substantially the same inflation slope as experienced in the latter portion of the second stage where it steepened, albeit later in time than either of the other two inflation slope lines relating to arteries 1 and 2.

And, in contrast to the right-most inflation line of artery 3, the left-most inflation line of artery 1 steepens immediately, or nearly immediately after crossing into the artery composition region. In contrast, the middle inflation line of artery 2 steepens also, but after a period of time has passed within the artery composition region.

In the third stage then the inflation slope changes based on the artery being dilated, the type of disease present thereon or therein, and the overall artery composition, including but not limited to the amount of calcification present.

### Fourth Stage

Here, the artery will yield if the inflation pressure (PSI) is not adequately controlled. If the artery yields, i.e., the resistance to expansion of the artery against the balloon drops significantly, the PSI will drop quickly and may experience a concomitant increase in volume. This becomes likely if the inflation line is allowed to proceed upward into the region marked “artery peak distention”.

The adaptive inflation and deflation device may react to different inflation slopes or conditions, i.e., first, second, third and/or fourth stage, or artery composition, or the type of artery being dilated, and/or the type of disease presented.

As seen in FIG. 9. “reaction windows” (shown in dashed lines bounding portions of the inflation line) may be provided which effectively mark out safe regions for the inflation line slope. There are 3 exemplary reaction windows, one corresponding with the inflation line of artery 1, one corresponding with the inflation line of artery 2 and one corresponding with the inflation line of artery 3. At least one of the reaction windows may change in width depending on the stage (first, second, third or fourth) the procedure or inflating balloon is within as described above.

Alternatively, a single reaction window may cover one, two or more stages.

Still more alternatively and preferably, as in the exemplary case of the middle inflation line corresponding with artery 2 as shown in FIG. 9, the reaction window may begin at a slope inflection point within a given stage and may extend to a subsequent slope inflection point, i.e., may extend between slope inflection points or may extend beyond at least one of two of more slope inflection points.

The length and width of the reaction window(s) may be stored within a memory for accessing, comparison and adaptive action by an operatively connected processor as described above.

For example, the inflation slope may break out of the designated boundaries of a predetermined reaction window on the high-pressure, low volume side of the associated and established reaction window. In adaptive response, the pressure at the inlet of the balloon may automatically adapt to be reduced, and/or the pressure within the balloon itself may be reduced (deflated). In the deflation adaptation case, the balloon may, in some cases, retract from contact so that dwell time may be reduced. Another reaction in this case might comprise adaptation by lowering or reducing the inflation rate of the balloon and bringing the inflation line back within the boundaries of the relevant reaction window.

Still another adaptation may comprise placing a hold on the inflation for a time, i.e., simply allowing the balloon to dwell for a time.

In some cases, the break-out (from the reaction window on the high-pressure, low volume left side) inflation line may be of a stage and/or have a slope that indicates a likely “yielding” of the subject artery. In this case, the adaptation will preferably be to immediately (1) stop inflation pressure and (2) to reduce pressure within the balloon.

Normally, as the balloon pressure increases, so does volume, causing in turn the balloon diameter to increase. But when the balloon is within a resistant or highly calcified lesion the pressure slope will begin to track at a higher slope and the volume slope will not increase along with pressure, nor will balloon diameter increase, so the volume slope flattens. When this happens. (slopes diverge) at a predetermined threshold level, embodiments of the present invention automatically adapt by, e.g., reducing the pressure amplitude or magnitude. Alternatively, or in addition, the automatic adaptation may also comprise changing the frequency or add dwell time between pressure increments.

Some exemplary embodiments of automated adaptive reactions to pressure, volume and/or inflation slope data breaking out of predetermined reaction window(s) follow.

The plot in FIG. 10 represents an exemplary balloon catheter inflation curve without resistance. This may be used to establish a baseline inflation pressure and volume and/or diameter plot and may be saved within the inflation device for a specific catheter for reference and/or comparison against a intravascular inflating balloon.

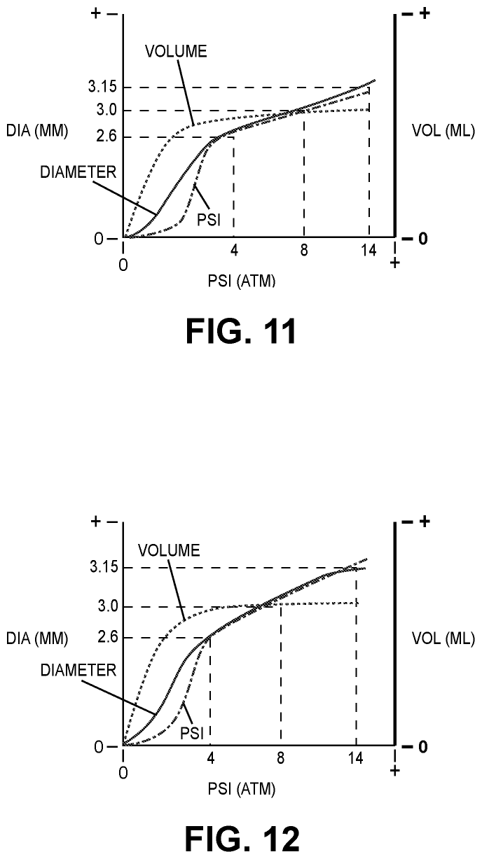

The plot in FIG. 11 represents the pressure and volume curve when the balloon catheter is inflated into a healthy artery. This plot could be defined as the plot that establishes specific artery compliance. This information from the actual compliance encountered would then pull up an incremental inflation routine that uses a predetermined or user selected pulse count with strain rate based on the artery compliance observed. Another method that could be used is what may be referred to as an integration inflation routine. That would be the first inflation of the balloon up to a safe 4-6 atm pressure, to establish the actual lesion compliance regardless of the healthy artery compliance.

The plot of FIG. 12 represents a simplified exemplary plot of what a high plaque burden and or calcified lesion inflation curve may provide. The more resistant the lesion is, the greater the slope differential is between, pressure and volume. It is here where the system becomes adaptive and readjusts the inflation algorithm based on real-time data of the actual lesion. Typically, the pressure pulse increments would be reduced (or increased) and the step rate goes up (or down). The reaction windows (not shown but as shown as in FIG. 9) would be +/− bands or a range of slopes would choose either a more or less aggressive total strain rates and or pulse and step frequency or amplitudes.

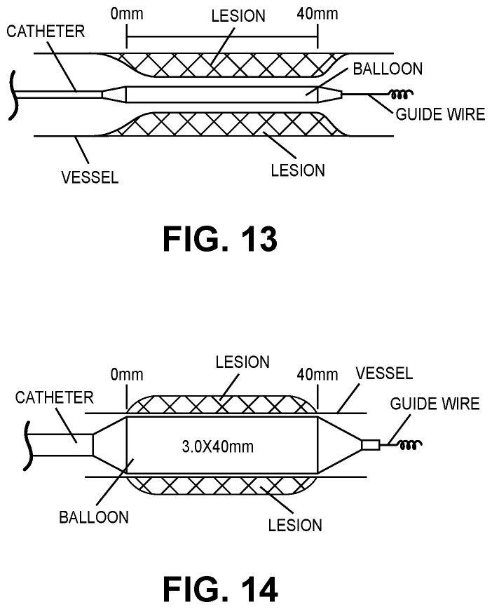

FIG. 13 illustrates a catheter with an angioplasty balloon and guidewire extending distally therefrom within a lesion within an blood vessel to be treated, with the cross-hatched area representing an exemplary area of a 3.0×40 mm lesion to be treated. The cross-hatched area make up the resistant lesion area that also would also be the reduction in volume of fluid needed to inflate just the balloon. This information provides the user a more accurate initial lesion size over the typical 2D angiogram.

FIG. 14 illustrates the balloon of FIG. 13 after the balloon is fully expanded to the exemplary area of 3.0×40 mm.

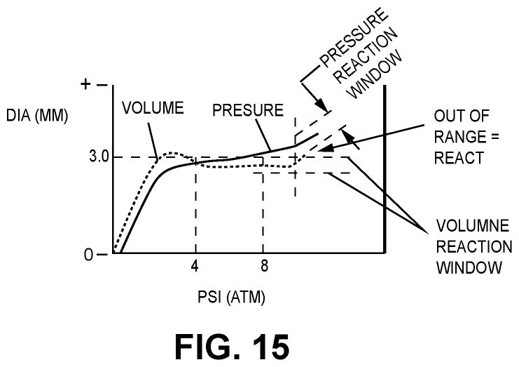

It is known that system (including but not limited to balloon) pressure will drop significantly when the lesion yields as described in connection with the fourth stage described above. FIG. 15 illustrates an exemplary yield situation. In this “yield” situation, the balloon diameter increases, lowering the pressure within the system to a point that is outside of the predetermined reaction window. When this happens when the artery sees a high strain rate usually near a resistant or calcified section. This is when the potential energy in the inflation device is converted to kinetic energy. Therefore, embodiments of the present invention monitor the system pressure and volume in real time and are configured to automatically react to “break outs” from predetermined reaction window boundaries by automatically adapting by, among other adaptions discussed herein, reducing the applied pressure (modifying plunger position), lowering the potential energy and lowering the strain rate the artery encounters. The monitored pressure and volume may be monitored within the balloon, at the plunger, or any other position along the fluid inflation/deflation pathway.

FIG. 15 also illustrates monitoring of the pressure and volume data and related slope of the resulting line. In certain embodiments, individual reaction windows may be established for the pressure and the volume separately. In this case, if the pressure breaks out of its pressure reaction window boundaries, or if the volume breaks out of its volume reaction window boundaries, the automatic adaptation of the system is triggered to correct the break out and bring the data back into the relevant reaction window. In other embodiments, a single reaction window may be established for the pressure and the volume, wherein a break out of either pressure or volume from the boundaries of the reaction window will trigger an automated adaptation of the system to bring the data back into compliance with the reaction window.

In addition, as described above, vessel tissue and/or material may be successively stretched. In this methodology, the pressure may be steadily increased for each pressure pulse period and/or may steadily increase within each pressure pulse period. Further, decompression periods between successive pressure pulse periods may be provided to allow the vessel tissue to relax and realign in preparation for the next successive pressure pulse period. The decompression periods may be zero or, preferably, may be greater than zero pressure. The foregoing may also be subjected to the automated adaptation described herein comprising predetermined reaction windows for inflation slope and/or pressure slope and/or volume slope, wherein breaking out of the reaction window(s) results in automatic adaptation by substantially immediate and automated pressure hold or reduction, decreasing (or otherwise modifying) pressure increment frequency, and/or add dwell time between pressure increments (either within pressure pulse periods, or between successive pressure pulse periods). In the case where dwell time is increased between successive pressure pulse periods, the decompression period is automatically modified to extend from a previous decompression period.

In certain embodiments, once an automated adaptation is executed, the resulting pressure and/or volume is sensed or measured and analyzed to determine whether the relevant slopes (inflation, pressure and/or volume) are now within the safe zone represented by the relevant reaction window. If the sensed or measured pressure and/or volume data moves back within the predetermined boundaries of the relevant reaction window, then the automated dilation process may continue, but at a modified (lower) pressure level with possible modification to other parameters as well, including but not limited to modifying pressure increment frequency and/or dwell time (for example but not limited to increasing decompression periods), changing, e.g., increasing, the length of relaxation periods between successive pressure pulse periods, changing the pressure pulse increment frequency, changing the pressure inflation velocity, and changing the pressure wave type.

Generally, all of the inflation and deflation methods, profiles and mechanisms described herein may be used within the context of the automated adaptation system comprising “safe” reaction windows.

Angioplasty balloons such as those known in the art have an unloaded diameter below which the balloon behaves a bladder such that the balloon pressure is substantially independent of volume and balloon pressure remains substantially zero below the unloaded diameter. When the balloon is constrained below its unloaded diameter, the the forces generated by the balloon pressure are transferred directly to the surrounding arterial structures. Once the balloon reaches its unloaded diameter, further increase in pressure causes balloon material to deform along with the surrounding arterial structures. Under these conditions a portion of the pressure energy is stored in the balloon material while the rest is applied to the lesion. It has also been observed that in some cases due to variation conditions along the length of the balloon a portion of the balloon may be above its unloaded diameter while other portions are below the unloaded diameter.

Precise characterization of the pressure, volume and flow characteristics of the catheter and inflation system permit arterial response to be measured. System characteristics useful for correcting, calibrating or compensating or solving for arterial response include: catheter shaft resistance, pump compliance, air bubble volume and/or compliance, seal compliance, balloon volume, balloon capacitance, unloaded balloon volume, creep, stress relaxation and viscoelastic behavior of the pump and balloon, plastic deformation of system components, stress softening of system components. In some embodiments System parameters, algorithms and correction factors are stored in a machine readable tag affixed to the catheter to enable device specific adaptive angioplasty algorithms to be applied by an inflation console.

In some embodiments, the reaction parameters are derived from dynamic pressure volume response. Signal processing and rheological methods useful for characterizing the dynamic response of the artery include Parameter estimation, system identification, impedance analysis, frequency analysis, impedance calibration, laplace transforms, solving of differential equations, integration and differentiation of functional measurements such as flow, pressure and volume over time.

Dynamic artery response can be characterized as: dPdV, Hysteresis of pressure—Volume curve, Storage modulus, loss modulus, complex impedance, resistance, reactance, time constant, settling time, response time, response rate, phase delay, response lag and other signal processing parameters.

In some embodiments, deviations from expected reaction windows are displayed to the user as warning, high/low indicator, normalized response indicator, plot, numerical display, audible of visual indicator. In other cases, deviations from the expected reaction windows cause a change in therapy delivery such as time, pressure, rate shape or manner of dilation. In still other embodiments deviations from the expected reaction window are used to suggest additional therapy such as re-dilatation with a larger balloon, atherectomy, stent, drug coated balloon, drug coated stent, and other treatment methods known in the art. In still other embodiments deviations from the expected reaction window may suggest alterations in medical therapy such as dual antiplatelet therapy.

The description of the invention and its applications as set forth herein is illustrative and is not intended to limit the scope of the invention. Features of various embodiments may be combined with other embodiments within the contemplation of this invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

We claim:

1. An automated adaptation inflation system for treating a blood vessel, comprising:
an inflatable balloon operatively connected to a catheter;
a fluid reservoir in operative fluid communication with the inflatable balloon;
a pressure controller in operative communication with the fluid reservoir and adapted to deliver fluid from the fluid reservoir with a plurality of pressure pulse periods, each pressure pulse period comprising at least one balloon inflation comprising at least one pressure magnitude, the pressure controller further adapted to provide a relaxation period between successive pressure pulse periods, each relaxation period comprising a balloon pressure that is greater than zero;
a processor in operative communication with the pressure controller, the processor comprising programmed instructions that, when executed, results in programmed execution of the plurality of pressure pulse periods and the relaxation period between successive pressure pulse periods;
a volume sensor configured to monitor the volume within the inflation system;
a pressure sensor configured to monitor the pressure within the inflation system;
wherein the programmed instructions of the processor comprise at least one reaction window comprising predetermined boundaries and configured to determine whether the monitored volume and/or pressure is: compliant and within the predetermined boundaries of the at least one reaction window, or non-compliant and outside of the predetermined boundaries of the at least one reaction window;
wherein the programmed instructions are configured to automatically adapt pressure applied to the balloon to bring a non-compliant monitored volume or pressure into compliance and within the predetermined boundaries of the at least one reaction window.

2. The system of claim 1, wherein the pressure is automatically adapted by at least one of the group consisting of: reducing pressure applied within the balloon, increasing dwell time, increasing the length of relaxation periods between successive pressure pulse periods, changing the pressure pulse increment frequency, changing the pressure inflation velocity, and changing the pressure wave type.

3. The system of claim 1, wherein each successive pressure pulse period comprises an increase in pressure magnitude.

4. The system of claim 1, wherein the pressure magnitude increases across each individual pressure pulse period.

5. A method for automated adaptive treatment of a blood vessel with an inflatable balloon catheter, comprising:
providing the inflatable balloon catheter;
a pressure controller in operative and fluid communication with a fluid reservoir and the inflatable balloon catheter;
a volume sensor in operative communication with the inflatable balloon catheter and/or the pressure controller and configured to monitor volume data in real time;
a pressure sensor in operative communication with the inflatable balloon catheter and/or the pressure controller and configured to monitor pressure data in real time;
a processor having programmed instructions, the processor in operative communication with the volume sensor, the pressure sensor, and the pressure controller,
wherein the programmed instructions comprise at least one reaction window having predetermined boundaries,
inserting the inflatable balloon catheter within the blood vessel to a location of interest;
using the pressure controller to execute a series of inflations;
receiving at the processor the monitored volume and pressure data;
determining if the monitored volume and pressure data are compliant within the predetermined boundaries of the at least one reaction window;
automatically adapting when at least one of the monitored and pressure data are non-compliant outside of the predetermined boundaries of the at least one reaction window; and
determining if the monitored volume and pressure data after the automatic adapting are compliant within the predetermined boundaries of the at least one reaction window.

6. The method of claim 5, wherein the automatic adapting comprises one or more of the group consisting of: reducing pressure applied within the balloon, increasing dwell time, increasing the length of relaxation periods between successive pressure pulse periods, changing the pressure pulse increment frequency, changing the pressure inflation velocity, and changing the pressure wave type.

7. The method of claim 6, further comprising providing relaxation periods between each inflation.

8. The method of claim 8, wherein each relaxation period comprises a balloon pressure magnitude greater than zero.

9. The method of claim 1, wherein each of the series of inflations comprises a pressure magnitude that increases with each successive inflation.

10. The method of claim 8, wherein each of the series of inflations comprises a pressure magnitude that increases with each successive inflation.

11. A method for calculating the initial inner diameter of a lesion within a blood vessel, and the final inner diameter post-procedure with an inflatable balloon catheter, comprising:
providing the inflatable balloon catheter;
a pressure controller in operative and fluid communication with a fluid reservoir and the inflatable balloon catheter;
a volume sensor in operative communication with the inflatable balloon catheter and/or the pressure controller and configured to monitor volume data in real time;
a pressure sensor in operative communication with the inflatable balloon catheter and/or the pressure controller and configured to monitor pressure data in real time;
a processor having programmed instructions, the processor in operative communication with the volume sensor, the pressure sensor, and the pressure controller;
determining using the monitored pressure data and the monitored volume data an initial inner diameter of the lesion;
executing a balloon angioplasty procedure to expand the lesion;
monitoring the inner diameter of the lesion using the monitored volume data and the monitored pressure data; and
determining, using the monitored volume data and the monitored pressure data, when the balloon angioplasty procedure is complete and further determining the inner diameter of the expanded lesion.

12. The method of claim 11, wherein the monitored volume data and monitored pressure data are monitored with each incremental pressure change.
